(19) United States
(12) Patent Application Publication
(10) Pub. No.: US 2020/0338313 A1
(43) Pub. Date: Oct. 29, 2020

(54) CATHETER PUNCTURE DEVICE

(71) Applicant: Bernd TIETZE, Adelsdorf (DE)

(72) Inventor: Bernd TIETZE, Adelsdorf (DE)

(21) Appl. No.: 16/912,407

(22) Filed: Jun. 25, 2020

Related U.S. Application Data
(63) Continuation of application No. 15/023,139, filed on Mar. 18, 2016.

(51) Int. Cl. A61M 25/06

(52) U.S. Cl. CPC A61M 25/0606; A61M 25/0693; A61M 2025/0008; A61M 25/0631; A61M 25/0618

(57) ABSTRACT

Catheter puncture device with a tubular housing, which merges into an extension piece for guiding the puncture needle that carries a blood collection container, abutment and blocking element, and from which a branch portion branches off through which a catheter can be pushed into the elongate housing portion, with a safety housing releasably connected to the tubular housing, wherein the puncture needle is guided on or in this safety housing, and a locking mechanism is provided for the puncture needle carrying a blood collection container and retracted after the puncture such that, after the puncture, the retraction of the puncture needle into the safety housing and the advance of the catheter into the tubular housing, it is possible for the safety housing, with the retracted puncture needle; locked therein and the blood collection container attached to the puncture needle, to be detached from the tubular housing.

### RELATED APPLICATIONS

This application is a continuation of, and claims priority to, U.S. patent application Ser. No. 15/023,139, titled “CATHETER PUNCTURE DEVICE” and filed on Mar. 18, 2016, which is a U.S. National Stage of PCT/IB2013/059536, filed Oct. 22, 2013, which are incorporated herein by reference.

### TECHNICAL FIELD

The invention relates to a catheter puncture set according to the preamble of claim 1.

### BACKGROUND

If a patient receiving medical care has to have a catheter inserted, then this is done by the medical staff usually using the so-called “Seldinger” technique. In a first step here, the vessel (often an artery or vein), into which the catheter is to be introduced, is punctured by a hollow puncture needle. Thereafter, a guide wire is pushed into the vessel through the hollow puncture needle. The hollow needle is then drawn back again over the inner guide wire, and therefore the guide wire can be advanced further into the punctured vessel in order to widen the latter to some extent in the first instance using a dilator which is pushed over the guide wire. Once the dilator has been removed, the actual catheter is pushed into the vessel over the horizontal guide wire and is displaced into the desired end position. As a final step, the guide wire is carefully drawn out again through the catheter.

This method for puncturing blood vessels using the Seldinger technique for the purpose of catheterization is, indeed, a standard method, but insertion of the catheter requires a large number of different maneuvers and instruments which presuppose much experience in this area. In addition, on account of various articles being pushed in and withdrawn again a number of times, there is a risk of articles or individuals being contaminated by escaping blood. Finally, there is also a risk of air embolism, specifically if there is a slight negative pressure prevailing in the punctured vessel. It is therefore known to have catheter puncture sets as catheterization aids, which are intended to simplify handling.

DE 101 00 102 discloses a catheter puncture set which simplifies the insertion of a catheter by providing the utensils which are necessary for puncturing and the insertion of a catheter in a system made up of a number of parts, said set therefore managing without a guide wire or additional dilator devices.

The catheter puncture set disclosed in said document comprises a tubular housing having an elongate housing portion which merges into an extension portion and from which a branch portion branches off at an angle. The housing portion and the branch portion have a lateral through-opening which is closed by a flexible casing, which extends along the branch portion and the housing portion and into which a catheter can be pushed. For puncturing of the vessel, the puncture needle, which extends along the elongate housing portion and the extension portion, exits out of the tip of the housing and, once puncturing has taken place, is drawn back into the housing to behind the branching-off location of the branch portion. A blood-collection container is located at the rear end of the puncture needle.

The catheter is then introduced to the desired depth into the punctured vessel from the housing, via the branch portion and the housing portion, through the flexible casing. It is only then that the catheter puncture set is withdrawn from the patient's punctured vessel. Finally, the flexible casing is withdrawn from the housing or peeled away from the catheter via a lateral longitudinal slot. Once puncturing has taken place using the catheter puncture set disclosed in DE 101 00 102, there is a total of two parts present in addition to the inserted catheter: on the one hand, the housing with the puncture needle and the blood-collection container; on the other hand, the detached casing.

In addition to this outlined prior art, there are also other methods known for catheter insertion using a modified Seldinger technique, some of the always multi-part catheter puncture sets using the so-called “peel-away” technique in which, once the catheter has been placed in the punctured vessel, the introduction sleeves can split into two parts on a “peel-away sheath” and be withdrawn from the vessel without the catheter being moved in the process.

### BRIEF DESCRIPTION OF THE DRAWINGS

To provide for a more detailed description, FIGS. 1 to 4 illustrate in detail an embodiment of the single-piece catheter puncture set according to the invention.

FIG. 1 shows a perspective illustration of the catheter puncture set according to the invention approximately in its original size,

FIG. 2 shows a perspective view of an enlarged detail prior to the puncturing step, with the puncture needle pushed in,

FIG. 3 shows a perspective view in which the safety housing 1 has been separated from the tubular housing 3, with the puncture needle 9 retracted into the safety housing 1, and

FIG. 4 shows a plan view of the safety housing 1 detached from the tubular housing 3.

### DETAILED DESCRIPTION

Against this background, it is an object of the present invention to create a catheter puncture set which further simplifies the insertion of a catheter in design terms and, at the same time, improves the handling thereof, wherein the number of individual components required for the catheter puncture set is reduced.

This is achieved by a catheter puncture set having the characterizing features of claim 1.

The dependent claims relate to advantageous configurations of the invention.

The catheter puncture set according to the invention constitutes a closed system which combines, and provides, in one device all the components necessary for puncturing purposes and, at the same time, makes it possible, on the one hand, to improve the handling of the catheter puncture set as a result of the structural components which are no longer required being detached following the puncture step and, on the other hand, to release the catheter from the rest of the structural components of the catheter puncture set and uncover the same, in one maneuver once the catheter has been introduced into the punctured vessel.

The core of the inventive idea is to simplify the insertion of the catheter in that, once the vessel has been punctured and the catheter has been introduced into the introduction sleeve of the catheter puncture set, the catheter puncture set can split into those structural components which are no longer required and those which are still necessary for introducing the catheter definitively into the punctured vessel.

This is achieved by the structural component constituting a safety housing, which, in the form of a central connecting component, connects the paired-by-design elements of the introduction sleeve and lateral-introduction catheter, on the one hand, to the puncture needle and the blood-collection container, on the other hand, wherein the safety housing can be assigned in design terms to the latter subassembly since, along with its function as a connecting member, it also serves for accommodating and securing the head of the puncture needle when the latter is retracted from the introduction sleeve once puncturing has taken place.

In the starting position of the catheter puncture set, here the safety housing is additionally a point of engagement on which to secure the blood-collection container with the puncture needle attached thereto. This means that, in order for the puncture needle to be secured against longitudinal displacement in the introduction sleeve when the vessel is punctured, the puncture needle, or the blood-collection container with the puncture needle attached, is fastened on the safety housing by means of a releasable connection.

This releasable connection between the blood-collection container or puncture needle and the safety housing can be established, for example, by a screw connection. However, it has been found to be particularly advantageous to provide an easily releasable snap-fit connection, which can be released by virtue of an arresting arm being raised, since the medical staff can thus quickly and readily release the connection and draw the puncture needle with the blood-collection container back out of the introduction sleeve until the puncture needle head strikes against a stop in the safety housing and latches in such that it is no longer possible for the puncture needle to be displaced out of the safety housing.

It is at this point, then, that the particular advantage of the safety housing according to the invention can be identified. This is because, if the puncture needles with the blood-collection container have been retracted from the introduction sleeve, the catheter can be pushed through the catheter puncture set. However, it has been found to be problematic that, in the prior art, the rearwardly projecting puncture needles with the blood-collection container are still connected to the catheter puncture set and therefore adversely affect the handling of the latter when the catheter is advanced and the inserted catheter is definitively separated from the catheter puncture set.

The safety housing is therefore designed such that it can be removed from the introduction sleeve with the catheter once the puncture needle has been retracted. The connection between the introduction sleeve and safety housing, accordingly is of releasable design, wherein it has been found to be advantageous, in a manner analogous to the above-described connection, to provide an easily releasable snap-fit connection, which can be released simply by virtue of an arresting arm being raised, since the medical staff can quickly and readily release the connection. Other, alternative easily releasable connecting means are also conceivable here.

As a result, as soon as the catheter has been advanced into the introduction sleeve, toward the blood vessel, and thus the blood rising up out of the blood vessel is channeled in the catheter, the safety housing can be unlocked and removed from the introduction sleeve containing the catheter. The puncture needle and the blood-collection container connected thereto are arrested here in the safety housing, as a result of which these can be fully removed from the catheter puncture set together with the safety housing and there is no possibility of the medical staff being injured on the puncture needle fixed in the safety housing. Following this step, the catheter puncture set therefore still comprises just the introduction sleeve with the catheter running therein. The task of handling these remaining elements of the catheter puncture set is correspondingly unproblematic.

In the final operating step, all that is then required is for the catheter to be advanced, through the introduction sleeve, into its end position in the blood vessel. At the same time, the introduction sleeve can be retracted from the blood vessel again. By virtue of at least one predetermined breaking location, for example a peel-away predetermined breaking location, along the introduction sleeve, it is then possible for the latter to be opened and removed from the catheter, which has then been definitively inserted.

In the prior art, the puncture needle projecting out to the rear and also the blood-collection container attached thereto constitute a very problematic extension to the catheter puncture set which, precisely at the critical moment of the catheter being pushed in, vastly restricts handling and thus causes misapplication and requires a certain amount of skill and practice. This drawback is eliminated altogether by the detachable safety housing according to the invention.

An advantageous embodiment of the catheter-puncture-set housing according to the invention is of just single-piece design, wherein the elongate housing portion, closed to form a tube and referred to as an introduction sleeve, both guides a puncture needle, which has its point exiting from the elongate housing portion and extends along the elongate housing portion and the extension portion, and at the same time, forms the guide channel for the catheter, which can be introduced into said guide channel via a branching-off location. Once the vessel has been punctured by the puncture needle and the puncture needle has been returned to behind the branching-off location of the branch portion, it is thus possible for the catheter to be introduced into the elongate housing portion via the branch portion, wherein at least one predetermined breaking location is arranged along the branch portion as far as the tip of the single-piece, elongate housing portion, said location allowing the housing to be opened, and therefore, once opening has taken place, the catheter is uncovered and the catheter puncture set can be removed.

The catheter puncture set according to the invention thus comprises, in a single introduction sleeve, in the first instance the puncture needle, which is pushed out of the elongate housing portion at one end and punctures the vessel. The end the introduction sleeve itself is also pushed a little way into the punctured vessel here. Once the blood vessel has been punctured, blood enters into the puncture needle and is accommodated in a preferably transparent blood-collection container arranged at the other end of the puncture needle.

The puncture needle is drawn back until the point ends up located in the safety housing behind the branching-off location of the branch portion of the housing and/or the introduction sleeve and a stop arranged on the puncture needle strikes against a corresponding abutment in the safety housing so that the puncture needle cannot be withdrawn all the way out of the catheter puncture set. In order for a situation where the puncture needle is pushed in anew likewise to be reliably avoided, a blocking element secures the puncture needle, and therefore the blood-contaminated point of the latter ends up located securely in the safety housing and does not pose any risk of injury to the medical staff or anyone else.

The catheter is then introduced into the punctured vessel, via the branch portion, by way of the housing and pushed into the vessel to the extent desired. In an advantageous embodiment, it is possible for the catheter to have applied to it markings which indicate to the medical staff how far the catheter should be advanced in the first step into the introduction sleeve and in the second step into the blood vessel. It is thus possible for the staff to identify reliably, with reference to the marking, that the catheter, for example, is already located in the blood vessel.

The introduced catheter, following detachment of the introduction sleeve, lies in an uncovered state and the catheter puncture set according to the invention, split into the introduction sleeve, on the one hand, and the safety housing together with the puncture needle and blood-collection container, on the other hand, can be disposed of easily and without posing any risk of injury.

In an advantageous embodiment of the invention, the catheter is fixed in a releasable manner, until it is pushed into the punctured vessel, on a holder arranged, in addition, on the housing. A clamping-action holder which can be released using one hand is expediently provided here. On the one hand, this ensures that, during the actual puncturing step, the flexible catheter does not form an obstruction or is not withdrawn from the puncture set. On the other hand, however, once puncturing has taken place, the catheter can easily be released from the fixing means and thus pushed into the punctured vessel.

A further advantageous configuration of the invention provides two approximately parallel predetermined breaking locations, which extend along the branch portion as far as the tip of the housing and/or of the introduction sleeve. It is advantageous here that the distance between said predetermined breaking locations defines the width of an opening strip which can be removed from the housing, wherein said width corresponds approximately to that of the catheter which is to be uncovered.

It is thus ensured that the task of drawing off the opening strip uncovers a slot-like opening over the entire guidance length of the catheter in the housing, and therefore the catheter can be released from the housing without force being applied. This is a significant improvement insofar as it can reliably prevent the situation where, when the housing is detached from the catheter pushed into the patient's vessel, the catheter is unintentionally withdrawn again from the vessel.

For the removal of this opening strip, provision is also made, in an expedient embodiment, for a tearing-open device arranged thereon in the region of the branch portion. This is advantageous insofar as the housing is opened along the predetermined breaking locations in the direction of, rather than away from, the patient undergoing the puncturing procedure. It is thus ensured, in turn, that the introduced catheter is not unintentionally withdrawn from the vessel, since the pulling movement to which the housing is subjected in order to remove the opening strip takes place in the direction of the patient.

In the case of an advantageous configuration of the catheter puncture set, a tearing-open device provided at the predetermined breaking location is one which is designed in the form of a 6gripping surface, on at least one side, for the improved introduction of tensile forces into the predetermined breaking location. By virtue of this protrusion-like gripping surface, of which the shape and size are configured for the gripping surface to be held securely for example between the index finger and thumb, being gripped, it is possible for the force required in order to overcome the breaking resistance of the for example notched predetermined breaking location to be introduced reliably.

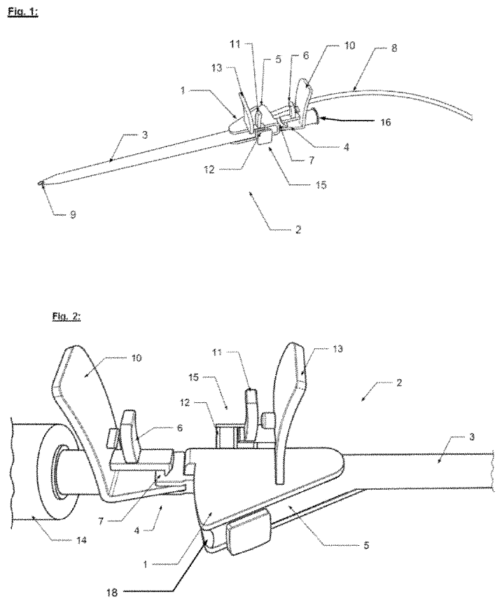

FIG. 1 shows the catheter puncture set 2 according to the invention in an illustration prior to a vessel being punctured. In this figure, there is no blood-collection container 14 plugged onto the corresponding connecting region 16 on the puncture needle 9.

The puncture needle 9 here has been introduced all the way into the tubular housing, and therefore the sharp-edged point of the puncture needle 9 exits from the front opening in the tubular housing 3. Two arresting regions 4 and 15 can be seen in this plugged-together version and also in FIG. 2. The arresting region 4 is formed from the movable arresting arm 6 and a needle guide 10, wherein the movable arresting arm 6 acts on a fastening portion 7, which in this embodiment is part of the safety housing 1. This arresting means 4 serves for fixing the puncture needle 9 in the safety housing 1 prior to, and during, the step of a vessel being punctured.

Once the vessel has been punctured, the two components of the movable arresting arm 6 and of the needle guide 10 are pressed onto one another, as a result of which the movable arresting arm 6 is raised and separated from the fastening portion 7. The puncture needle 9 can then be gripped by the needle guide 10 and drawn back out of the tubular housing 3 into an arresting position in the safety housing 1, in which the point of the puncture needle 9 latches in the safety housing 1 and there is therefore no longer any risk of the medical staff being injured.

The second arresting portion 15 is relevant to the connection between the tubular housing 3 and the safety housing 1. In the present embodiment, this arresting means 15 is also formed from an arresting arm 11 and a retaining plate 13, wherein the arresting arm 11, in a connection position of the components, engages in a mount 12 which, in the exemplary embodiment illustrated, is part of the tubular housing 3. It is a variable matter here, however, as to whether the arresting arm 11 is part of the tubular housing 3 and fastening takes place on the safety housing 1, or vice versa. The critical factor is that releasable arresting takes place here, it being possible for the arresting to be released, for example, as a result of the arresting arm 11 and retaining plate 13 being pressed together.

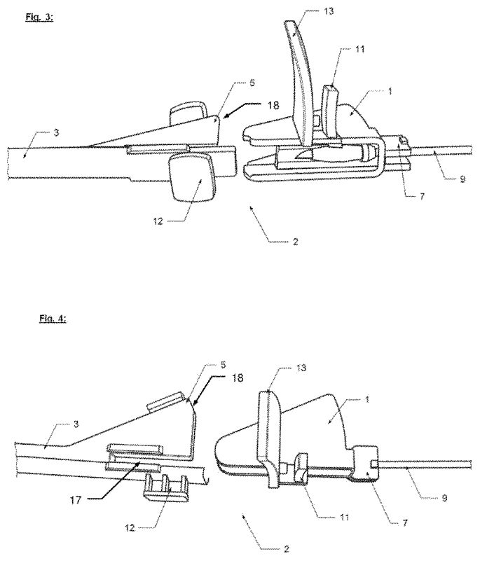

The separated components made up of the tubular housing 3 and safety housing 1 can be seen in FIGS. 3 and 4. In particular FIG. 4 illustrates that the safety housing 1 can be pushed into a corresponding accommodating opening 17 in the tubular housing 3 until the movable arresting arm 11 latches into the mount 12 and the two components are therefore securely connected to one another.

It can also be seen in FIG. 3 that, here, the puncture needle 9 has already been retracted into its arresting position in the safety housing 1. In this position, in the branch portion 5, the catheter 8 has already been advanced into the tubular housing 3 or the punctured vessel and, at the same time, the puncture needle 9 has been retracted through the tubular housing 3. The course taken by the catheter 8 through the branch portion 5 can be seen here merely in FIG. 1, in which the catheter 8 has not yet been pushed into the tubular housing 3 since, here, the puncture needle 9 is still located in the tubular housing 3. There is deliberately no provision made for parallel guidance of the two components in the tubular housing 3.

Once the puncture needle 9 has been drawn back out of the tubular housing 3, the catheter 8 is advanced and can thus be introduced cleanly into the vessel. It is a decisive advantage of the present inventive solution here that, as advancement of the catheter 8 into the punctured vessel continues, it has already been possible for the safety housing 1 with the puncture needle 9 secured therein, and the blood-collecting container 14 attached to the puncture needle, to be removed from the tubular housing 3 and set aside. It has been found in practice that the task of pushing the catheter 8 in by way of the catheter puncture set 2 is obstructed by the puncture needle 9 with blood-collection container 14 still being located on the catheter puncture set 2, and this results, relatively frequently, in errors during the insertion of the catheter 8.

In the present inventive solution, all that is required is for the catheter 8 to be advanced some what when the puncture needle 9 is withdrawn from the tubular housing 3, and therefore the tubular housing 3 is filled with the catheter 8 and the rising blood thus penetrates into the catheter 8. It is at this point that the corresponding fastening mechanisms allow the safety housing 1 to be removed from the tubular housing 3, as a result of which these components no longer obstruct the continued advancement of the catheter 8.

FIG. 2 shows the lateral view of the tubular housing 3, it being possible to see the mount 18 for the catheter 8 in the branch portion 5. It is relevant to the solution according to the invention here that it is possible here for said tubular housing 3 to be opened via at least one longitudinally extending predetermined breaking location (not illustrated in the drawings) in the form of a peel-off strip, and therefore, once the catheter 8 has been inserted, the tubular housing 3 can be opened by way of said corresponding predetermined breaking location and drawn off from the catheter 8 without the inserted catheter 8 being unintentionally withdrawn from the punctured vessel.

What is claimed is:

1. A method of inserting a catheter using a catheter puncture set, the catheter puncture set comprising a tubular housing, a puncture needle, and a catheter, the tubular housing having an elongate housing portion which merges into an extension portion for guiding the puncture needle and from which a branch portion branches off at an angle, the branch portion in communication with the elongate housing portion and configured to receive the catheter from the branch portion into the elongate housing portion, wherein a safety housing is connected in a releasable manner to the tubular housing, wherein the safety housing is configured to guide the puncture needle through a front opening in the tubular housing when performing a puncture; a first movable arresting arm connected with the safety housing is provided for a first arresting region between the puncture needle and the safety housing; and a second movable arresting arm is provided for a second arresting region between the safety housing and the tubular housing, the method comprising the steps of:
placing the front opening of the tubular housing proximate to a patient's blood vessel;
guiding the puncture needle through a front opening in the tubular housing to puncture the blood vessel;
releasing the first movable arresting arm and retracting the puncture needle into the safety housing;
releasing the second movable arresting arm and detaching the safety housing from the tubular housing;
inserting the catheter through the tubular housing into the vessel; and
removing the tubular housing from the catheter.

2. The method of claim 1, wherein the puncture needle is retracted into the safety housing where the retracted puncture needle is arrested.

3. The method of claim 1, wherein the safety housing along with the retracted puncture needle arrested therein is removed from the tubular housing.

4. The method of claim 1, wherein the first movable arresting arm fixes the puncture needle in a releasable manner in a pushed-in puncturing position on the safety housing.

5. The method of claim 1, wherein the first movable arresting arm is released when the first movable arresting arm and needle guide are pressed together.

6. The method of claim 1, wherein the second movable arresting arm is connected to the safety housing and is configured to engage in a mount on the tubular housing or act on a fastening portion on the tubular housing, wherein this connection is releasable by virtue of the second movable arresting arm being guided up to a handle-like retaining plate on the safety housing as a result of the second movable arresting arm and the handle-like retaining plate being pressed together.

7. The method of claim 1, wherein the second movable arresting arm being connected to the tubular housing engages a mount on the safety housing or acts on a fastening portion of the safety housing, wherein this connection is released by virtue of the second movable arresting arm being guided up to a handle-like retaining plate on the tubular housing as a result of the second movable arresting arm and the handle-like retaining plate being pressed together.

8. The method of claim 1, wherein the tubular housing of the catheter puncture set is of a single-piece and closed design, wherein the elongate housing portion, closed to form a tube, both guides the puncture needle, which has its point exiting from the front opening of the elongate housing portion and extends along the housing, and, at the same time, forms the guide channel for the catheter to be introduced into the elongate housing portion via the branch portion.

9. The method of claim 1, wherein the first movable arresting arm fixes the puncture needle in a releasable manner in a pushed-in puncturing position within the tubular housing.

10. The method of claim 9, wherein the first movable arresting arm arrests the puncture needle, the first movable arresting arm engages a mount or acts on a fastening portion on the tubular housing, wherein the first movable arresting arm is released when the first movable arresting arm is guided up to a needle guide as a result of the first movable arresting arm and needle guide being pressed together.

11. A method of inserting a catheter into a vessel using a catheter puncture set comprising a tubular housing, a puncture needle, and a catheter, the tubular housing having an elongate housing portion which merges into an extension portion for guiding the puncture needle and from which a branch portion branches off at an angle, the branch portion in communication with the elongate housing portion and configured to receive the catheter from the branch portion into the elongate housing portion, wherein a safety housing is connected in a releasable manner to the tubular housing, wherein the safety housing is configured to guide the puncture needle through a front opening in the tubular housing; a first movable arresting arm connected with the safety housing is provided for a first arresting region between the puncture needle and the safety housing, wherein the first movable arresting arm fixes the puncture needle in a releasable manner prior to and during a puncture step, a second movable arresting arm is provided for a second arresting region between the safety housing and the tubular housing, wherein the second movable arresting arm connects the safety housing and the tubular housing in a releasable manner, wherein the second movable arresting arm is connected to the safety housing and is configured to engage in a mount on the tubular housing or act on a fastening portion on the tubular housing, wherein this connection is releasable by virtue of the second movable arresting arm being guided up to a handle-like retaining plate on the safety housing as a result of the second movable arresting arm and the handle-like retaining plate being pressed together; the method comprising the steps:
puncturing a vessel with a puncture needle;
retracting the puncture needle into the safety housing where the retracted puncture needle is arrested;
removing the safety housing and retracted puncture needle from the tubular housing;
advancing the catheter through the tubular housing and into the vessel; and
removing the tubular housing from the catheter.

12. The method of claim 11, wherein the second movable arresting arm is connected to the tubular housing and engages a mount on the safety housing or acts on a fastening portion of the safety housing, wherein this connection is released by virtue of the second movable arresting arm being guided up to a handle-like retaining plate on the tubular housing as a result of the second movable arresting arm and the handle-like retaining plate being pressed together.

13. A method of inserting a catheter into a vessel using a catheter puncture set comprising a tubular housing, a puncture needle, and a catheter, the tubular housing having an elongate housing portion which merges into an extension portion for guiding the puncture needle and from which a branch portion branches off at an angle, the branch portion in communication with the elongate housing portion and configured to receive the catheter from the branch portion into the elongate housing portion, wherein a safety housing is connected in a releasable manner to the tubular housing, wherein the safety housing is configured to guide the puncture needle through a front opening in the tubular housing; a first movable arresting arm connected with the safety housing is provided for a first arresting region between the puncture needle and the safety housing, wherein the first movable arresting arm fixes the puncture needle in a releasable manner prior to and during the puncture step, a second movable arresting arm is provided for a second arresting region between the safety housing and the tubular housing, wherein the second movable arresting arm connects the safety housing and the tubular housing in a releasable manner, wherein the second movable arresting arm being connected to the tubular housing and configured to engage in a mount on the safety housing or act on a fastening portion of the safety housing, wherein this connection is releasable by virtue of the second movable arresting arm being guided up to a handle-like retaining plate on the tubular housing as a result of the second movable arresting arm and the handle-like retaining plate being pressed together; the method comprising:
puncturing a vessel using a puncture needle;
retracting the puncture needle into the safety housing;
arresting the puncture needle within the safety housing;
removing the safety housing from the tubular housing;
advancing a catheter through the tubular housing and into the vessel; and
removing the tubular housing from the catheter.

14. The method of claim 13, wherein the second arresting region is at a distal end of the safety housing from the tubular housing.

15. The catheter puncture set as claimed in claim 13, wherein the second arresting region comprises a mount over which the first movable arresting arm is configured to move to retain the needle guide connected with the safety housing.

16. The method of claim 13, wherein the safety housing further comprises a retaining plate, the first movable arresting arm is configured to disengage from the second arresting region in response to movement of the first movable arresting arm toward the retaining plate.

17. The method of claim 13, wherein the safety housing further comprises a retaining plate, the second movable arresting arm is configured to disengage from the first arresting region in response to movement of the second movable arresting arm toward the retaining plate.

18. The method of claim 13, wherein the first arresting region comprises a mount with which the second movable arresting arm is configured to engage to retain the safety housing connected with the tubular housing.

19. The method of claim 13, wherein the tubular housing has an opening slot extending longitudinally from an open end of the tubular housing, at least a portion of the safety housing is configured to fit into the opening slot.

20. The method of claim 13, wherein the tubular housing has an opening slot extending longitudinally from an open end of the tubular housing, the second movable arresting arm is configured to engage with the first arresting region when the safety housing is positioned in the opening slot.
